(19) United States
(12) Patent Application Publication
(10) Pub. No.: US 2025/0018178 A1
(43) Pub. Date: Jan. 16, 2025

(54) Medical Electrode Device Comprising at Least One Contact Element

(71) Applicant: BIOTRONIK SE & Co. KG, Berlin (DE)

(72) Inventors: Patrick Willenberg, Dissen-Striesow (DE); Dajana Kaiser, Berlin (DE); Kerstin Taeubert, Berlin (DE); Tobias Mueller, Berlin (DE); Thomas Schaarschmidt, Schulzendorf (DE); Roland Eichberg, Berlin (DE)

(73) Assignee: BIOTRONIK SE & Co. KG, Berlin (DE)

(21) Appl. No.: 18/713,982

(22) PCT Filed: Oct. 28, 2022
(86) PCT No.: PCT/EP2022/080187

§ 371 Date: May 28, 2024

(30) Foreign Application Priority Data
Dec. 3, 2021 (EP) 21212321.0

(51) Int. Cl. A61N 1/05

(52) U.S. Cl. CPC A61N 1/0553

(57) ABSTRACT

A medical electrode device for implantation into a patient comprises a carrier element formed from an electrically insulating material, a contact element arranged on the carrier element and comprising a contact portion for coupling to tissue in an implanted state of the medical electrode device, and an electrical supply line connected to the contact element. The contact element comprises first and second fastening sections, the first and second fastening sections arranged at a distance with respect to each other along a length direction, and the electrical supply line connected to the contact element at the first fastening section and the second fastening section such that the electrical supply line is arranged at a distance with respect to the contact portion along a height direction oriented perpendicularly to the length direction. A portion of the carrier element extends through a space in between the contact portion and the electrical supply line.

### CROSS-REFERENCE TO RELATED APPLICATIONS

This application is the United States National Phase under 35 U.S.C. § 371 of PCT International Patent Application No. PCT/EP2022/080187, filed on Oct. 28, 2022, which claims the benefit of European Patent Application No. 21212321.0, filed on Dec. 3, 2021, the disclosures of which are hereby incorporated by reference herein in their entireties.

### TECHNICAL FIELD

The present invention relates to a medical electrode device for implantation into a patient according to the preamble of claim 1 and to a method for fabricating a medical electrode device.

### BACKGROUND

A medical electrode device of this kind may, for example, serve for a neuro-stimulation and for this may be implanted into a patient, for example, in the region of the spinal cord, for example into the epidural space near the spinal cord of the spinal column of a patient. In this way a nerve stimulation at the spinal cord may be achieved by injecting electrical stimulation currents.

An electrode device of the kind concerned herein however may also be used for emitting stimulation signals or receiving sense signals at other locations within a patient, for example within the brain or in cardiac applications.

An electrode device of the kind concerned herein comprises a carrier element being formed from an electrically insulating material, at least one contact element arranged on the carrier element and comprising a contact portion for coupling to tissue in an implanted state of the medical electrode device, and at least one electrical supply line connected to the at least one contact element. The at least one contact element comprises a first fastening section and a second fastening section, the first fastening section and the second fastening section being arranged at a distance with respect to each other along a length direction and the at least one electrical supply line being connected to the at least one contact element at the first fastening section and the second fastening section such that the at least one electrical supply line is arranged at a distance with respect to the contact portion along a height direction oriented perpendicularly to the length direction.

The medical electrode device may have the shape of a so-called paddle electrode, the carrier element in this case having a flattened, paddle-like shape carrying, e.g., an arrangement of multiple evenly or unevenly distributed contact elements on its surface for emission of electrical signals into and/or reception of electrical signals from surrounding tissue.

There is a general desire to enable fabrication of a medical electrode device in an easy and cost-efficient manner.

In addition, there is a desire to provide a medical electrode device in which contact elements are arranged on a carrier element in a locationally stable manner.

U.S. Pat. No. 6,052,608 discloses an electrode device used in particular for sensing cortical electrical activity. The electrode device herein comprises an arrangement of contact elements having a semi-spherical shape, the contact elements being surrounded by an electrically insulating material of a carrier element.

Different designs of paddle electrodes are known, for example, from U.S. Pat. Nos. 6,895,283 and 9,561,363.

European Publication No. 2 108 398 A1 discloses an implantable medical lead comprising a first paddle portion having a cavity and a second paddle portion having at least one aperture. At least one electrode is electrically connected to at least one conductor. The at least one electrode is placed in the cavity of the first paddle portion and is disposed between the first paddle portion and the second paddle portion. The at least one electrode is disposed in such a way that a portion of the electrode is conductively exposed through the at least one aperture.

U.S. Publication No. 2008/0046050 A1 and U.S. Publication No. 2014/0172057 A1 disclose other medical electrode devices in the shape of paddle electrodes.

The present disclosure is directed toward overcoming one or more of the above-mentioned problems, though not necessarily limited to embodiments that do.

### SUMMARY

It is an object of the instant invention to provide a medical electrode device and a method for fabricating a medical electrode device allowing for an easy and cost-efficient fabrication as well as an improved mechanical stability of one or multiple contact elements on a carrier element.

At least this object is achieved by means of a medical electrode device comprising the features of claim 1.

Accordingly, a portion of the carrier element extends through a space in between the contact portion and the at least one electrical supply line.

The medical electrode device comprises one or multiple contact elements arranged on a carrier element. One or multiple contact elements for this are at least partially embedded in the electrically insulating material of the carrier element, wherein a contact portion of each contact element is exposed towards the outside in order to enable a coupling of the contact elements to surrounding tissue in an implanted state of the electrode device.

The carrier element in particular may have a flattened, paddle-like shape. The carrier element may be arranged on a lead body by means of which the carrier element with the contact elements arranged thereon is connected to, e.g., a generator implanted in a patient or placed outside of a patient (in an implanted state of the implantable electrode device). The carrier element herein in particular may be arranged at a distal end of the lead body, the lead body at its proximal end being connected to the generator, for example by means of a suitable connector.

For supplying an electrical current to the at least one contact element, the at least one contact element is connected to at least one electrical supply line. For this, the contact element comprises a first and a second fastening section, the fastening sections serving to establish a connection between the associated supply line and the contact element, wherein the connection may be a direct connection—in which the supply line directly is placed on the fastening sections—or may be an indirect connection—in which the supply line, for example, is received on and connected to a connection element, which is connected to the fastening sections.

The fastening sections are displaced with respect to one another along a length direction, such that the fastening sections are arranged at a distance with respect to one another along the length direction. In addition, the fastening sections are displaced with respect to the contact portion of the contact element (by means of which an electrical coupling to surrounding tissue is established in an implanted state of the electrode device), such that the electrical supply line is arranged at a distance with respect to the contact portion along a height direction oriented perpendicularly to the length direction. A space hence is formed in between the fastening sections (along the length direction) and between the electrical supply line and the contact portion (along the height direction perpendicular to the length direction).

In order to improve a locational stability of the contact element on the carrier element it herein is proposed to use the space formed between the electrical supply line and the contact portion of the contact element to provide an additional engagement between the carrier element and the contact element. Because the electrical supply line (directly or indirectly) is connected to the fastening sections and extends at a distance to the contact portion of the contact element, an opening is formed, the opening being bound by the fastening sections, the electrical supply line and the contact portion. By having a portion of the carrier element extend through that opening in between the fastening sections and in between the electrical supply line and the contact portion, the carrier element lockingly engages with the contact element in connection with the electrical supply line, such that the mechanical stability and locational fixation of the contact element on the carrier element is improved.

As shall be explained in further detail below, the portion of the carrier element may be a portion of the insulating material of the carrier element. Alternatively or in addition, a reinforcement structure of the carrier element such as a reinforcement layer made of a woven fabric may extend through the space in between the contact portion and the at least one electrical supply line in a range between the first fastening section and the second fastening section, wherein the reinforcement structure may be embedded in the insulating material of the carrier element.

In one embodiment, the contact portion faces outside in a first direction along the height direction, wherein the at least one contact element comprises an edge contour facing in a second direction opposite the first direction. The first fastening section and the second fastening section herein are formed on the edge contour. The contact portion may, for example, have a flat or curved shape in order to establish an electrical contact to surrounding tissue. The contact portion faces outside and hence is exposed towards the outside such that the contact portion, in an implanted state of the medical electrode device, may be brought into contact with the surrounding tissue. The contact element may be, for example, formed from a metal material, for example by employing a deep drawing process, wherein the edge contour bounding the contact element, for example, is displaced along the height direction with respect to the contact portion.

The fastening sections may be formed by portions of the edge contour, such that the electrical supply line or a connection element for receiving the electrical supply line may be placed on and may be connected to edges of the edge contour, for example by welding.

In another embodiment, at least one of the first fastening section and the second fastening section may protrude from (other portions of) the edge contour along the second direction, i.e., in a direction pointing away from the contact portion (along the height direction). The first fastening section and the second fastening section, in this embodiment, may, for example, have the shape of a flap or a tongue extending from the edge contour, the first fastening section and the second fastening section being configured to establish a connection to the electrical supply line or a connection element for receiving the electrical supply line.

In one embodiment, the at least one contact element comprises at least one engagement element protruding from (other portions of) the edge contour and engaging with the electrically insulating material of the carrier element. The engagement element may, for example, serve to provide for an additional engagement with the insulating material of the carrier element, such that by means of the engagement element the locational stability of the contact element within the insulating material of the carrier element is further improved. The engagement element may, for example, have a wing-like shape, the engagement element extending from the edge contour in a direction pointing away from the contact portion, wherein the engagement element may, for example, be curved in its cross-sectional shape.

In one embodiment, the first fastening section and the second fastening section are formed on a first portion and a second portion of the edge contour arranged at a distance with respect to each other along the length direction. Herein, the at least one engagement element is arranged on a third portion of the edge contour extending in between the first portion and the second portion along the length direction, such that the engagement element is arranged to extend in between portions of the edge contour carrying the fastening sections. Two engagement elements herein may be arranged on opposing portions of the edge contour, the engagement elements being formed and arranged symmetrically on the contact element.

In one embodiment, at least one of the first fastening section and the second fastening section comprises a fastening opening in which the at least one electrical supply line or a connection element receiving the electrical supply line is arranged. Generally, the electrical supply line or a connection element receiving the supply line may be arranged on edges of the edge contour of the contact element, portions of the edge contour hence forming the fastening sections. To improve the mechanical stability and the precision of the seating of the electrical supply line or the connection element receiving the electrical supply line on the fastening sections, herein, a fastening opening may be formed on the respective fastening section, the fastening opening being such that the electrical supply line or the connection element receiving the electrical supply line may be received within the fastening opening.

In one embodiment, the fastening opening is circumferentially closed. The fastening opening hence is formed by an opening having, for example, a circular shape, e.g., in a plane perpendicular to the length direction.

In another embodiment, the fastening opening is circumferentially opened, the fastening opening, for example, having a semicircular shape, e.g., in a plane perpendicular to the length direction, such that the electrical supply line or the connection element receiving the electrical supply line may be placed in the fastening opening in a direction perpendicular to the length direction. The fastening opening, in this embodiment, is formed by a recess configured to receive the electrical supply line or a connection element connected to the electrical supply line therein.

In one embodiment, the medical electrode device comprises a connection element configured to connect the at least one electrical supply line to the first fastening section and the second fastening section. The connection element herein is fastened on the first fastening section and the second fastening section. By means of the connection element the electrical supply line is arranged on and fixed to the contact element, such that an indirect fixation of the electrical supply line on the contact element is established.

The connection element, in one embodiment, may comprise a conduit in which the at least one electrical supply line is received and within which the electrical supply line is connected to the connection element. The connection element, for this, may, for example, have the shape of a longitudinal sleeve extending along the length direction, the connection element, for example, being connected to the first fastening section and the second fastening section by means of welding, such that the longitudinal connection element extends in between the first fastening section and the second fastening section.

The connection element may, for example, be a crimp sleeve allowing to establish a crimp connection in between the connection element and the electrical supply line received in the connection element.

The connection between the connection element and the electrical supply line may be established prior to or after the connection element is connected to the contact element. By connecting the electrical supply line to the connection element after the connection element has been connected already to the contact element, it may, for example, become possible to adjust a position of the electrical supply line with respect to the connection element and hence with respect to the contact element, allowing to arrange the electrical supply line in between the lead body and the contact element in an easy and space efficient manner, while avoiding a strain on the electrical supply line due to its connection to the contact element.

In another embodiment, the connection element comprises a plate body configured to fasten the at least one electrical supply line to the contact element. The plate body may have the shape of a rectangular plate and may extend flatly along a plane perpendicular to the height direction.

The plate body may be used, for example, to weld the electrical supply line to the plate body, such that the electrical supply line is (indirectly) connected to the fastening sections when the plate body is arranged on the fastening sections. Alternatively, the plate body may be used to secure the electrical supply line on the fastening sections, for example by clamping the electrical supply line to the fastening sections.

In one embodiment, the at least one supply line is arranged on a side of the plate body facing away from the contact portion. In this embodiment the electrical supply line may, for example, be welded to the side of the plate body facing away from the contact portion.

In another embodiment, the at least one electrical supply line is arranged on a side of the plate body facing towards the contact portion. In this embodiment, the at least one electrical supply line may be welded to the plate body, or may be received in openings on the fastening sections and may be clamped to the fastening sections by means of the plate body.

In one embodiment, the portion of the carrier element extending through the space in between the electrical supply line and the contact portion of the contact element is formed by a portion of the electrically insulating material of the carrier element, which, e.g., during a molding process may be caused to flow into and through the space formed in between the electrical supply line and the contact portion of the contact element in a range between the fastening sections.

In another embodiment, the portion of the carrier element extending through the space in between the electrical supply line and the contact portion is a reinforcement layer embedded in the electrically insulating material of the carrier element. The reinforcement layer may, for example, be made from a woven fabric, e.g., a textile fabric, the reinforcement layer improving the mechanical durability and form stability of the carrier element. By extending through the space in between the electrical supply line and the contact portion, the reinforcement layer further supports the contact element on the carrier element and hence improves the mechanical connection in between the carrier element and the contact element.

In another aspect, a method for fabricating a medical electrode device for implantation into a patient comprises: forming a carrier element from an electrically insulating material such that at least one contact element is at least partially embedded in the electrically insulating material of the carrier element, the at least one contact element comprising a contact portion for coupling to tissue in an implanted state of the medical electrode device; and connecting at least one electrical supply line to the at least one contact element, wherein the at least one contact element comprises a first fastening section and a second fastening section, the first fastening section and the second fastening section being arranged at a distance with respect to each other along a length direction and the at least one electrical supply line being connected to the at least one contact element at the first fastening section and the second fastening section such that the at least one electrical supply line is arranged at a distance with respect to the contact portion along a height direction oriented perpendicularly to the length direction. Herein, said forming the carrier element includes: forming the carrier element such that a portion of the carrier element extends through a space in between the contact portion and the at least one electrical supply line.

The advantages and advantageous embodiments described above for the medical electrode device equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a view of an electrode device connected to a stimulation device in an implanted state in the area of the spine of a patient;

FIG. 2 shows a view of the electrode device in the epidural space in the region of the spinal column:

FIG. 3 shows a view of a flattened end of an embodiment of an electrode device:

FIG. 4 shows a view of an electrode device with contact elements arranged on a flattened end of the electrode device;

FIG. 5 shows a cross-sectional view of a contact element in a carrier element of an electrode device;

FIG. 6A shows a view of a contact element;

FIG. 6B shows the contact element of FIG. 6A, with a connection element for receiving an electrical supply line arranged on the contact element;

FIG. 7A shows a view of another embodiment of a contact element;

FIG. 7B shows the contact element of FIG. 7A, with a connection element for receiving an electrical supply line arranged on the contact element;

FIG. 8A shows a view of yet another embodiment of a contact element;

FIG. 8B shows the contact element of FIG. 8A, with a connection element for receiving an electrical supply line arranged on the contact element;

FIG. 9A shows a view of yet another embodiment of a contact element;

FIG. 9B shows the contact element of FIG. 9A, with a connection element for receiving an electrical supply line arranged on the contact element;

FIG. 10A shows a view of yet another embodiment of a contact element;

FIG. 10B shows the contact element of FIG. 10A, with a connection element for receiving an electrical supply line arranged on the contact element;

FIG. 11A shows a view of yet another embodiment of a contact element;

FIG. 11B shows the contact element of FIG. 11A, with a connection element for receiving an electrical supply line arranged on the contact element;

FIG. 12A shows a view of another embodiment of a contact element;

FIG. 12B shows the contact element of FIG. 12A, with an electrical supply line (directly) arranged on the contact element;

FIG. 13A shows a view of yet another embodiment of a contact element;

FIG. 13B shows the contact element of FIG. 13A, with an electrical supply line (directly) arranged on the contact element;

FIG. 14A shows a view of yet another embodiment of a contact element;

FIG. 14B shows the contact element of FIG. 14A, with an electrical supply line (directly) arranged on the contact element;

FIG. 15A shows a view of another embodiment of a contact element;

FIG. 15B shows the contact element of FIG. 15A, with a connection element in the shape of a plate body connected to the contact element;

FIG. 16A shows a view of yet another embodiment of a contact element;

FIG. 16B shows the contact element of FIG. 16A, with a connection element in the shape of a plate body connected to the contact element;

FIG. 17A shows a view of yet another embodiment of a contact element;

FIG. 17B shows the contact element of FIG. 17A, with a connection element in the shape of a plate body connected to the contact element:

FIG. 18A shows a view of yet another embodiment of a contact element:

FIG. 18B shows the contact element of FIG. 18A, with a connection element in the shape of a plate body connected to the contact element and clamping an electrical supply line to the contact element:

FIG. 19A shows a view of a positioning tool during a manufacturing step for assembling the contact elements, the connection elements and the electrical supply lines of the electrode device; and

FIG. 19B shows the arrangement of FIG. 19A, with contact elements, a reinforcement layer and connection elements received in the positioning tool for carrying out a assembly process.

### DETAILED DESCRIPTION

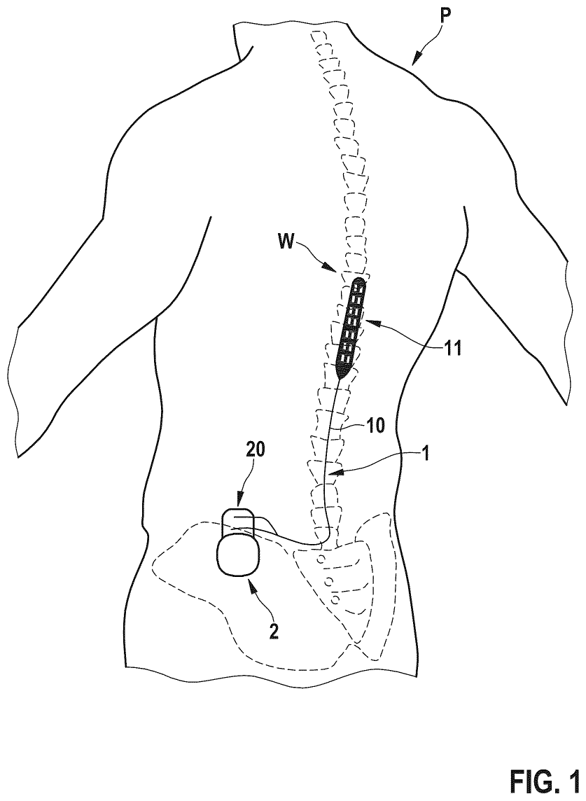

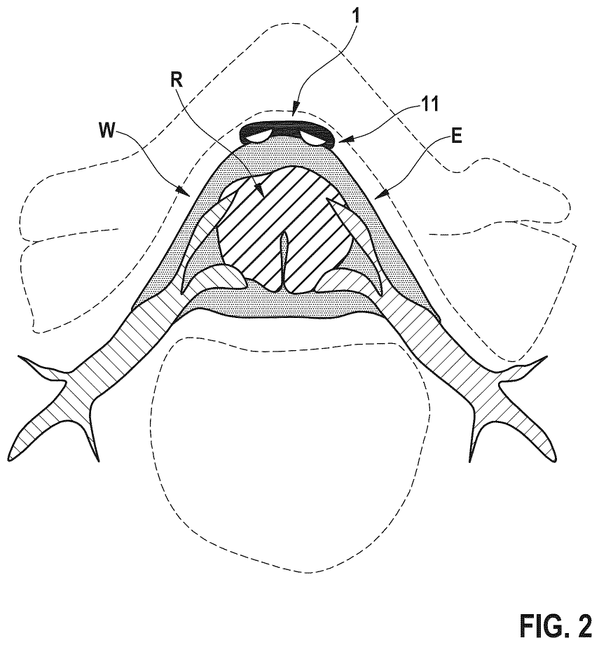

An electrode device 1, as shown in an embodiment in FIGS. 1 and 2, is formed as a so-called paddle electrode and comprises a lead body 10 and an electrode end 11 connected to the lead body 10, a plurality of contact elements being attached to the electrode end 11 for injecting an electrical current, e.g., in the region of the spinal column W of a patient P.

The electrode device 1 at a proximal end of the lead body 10 is connected to a connector block 20 of a stimulation device 2, via which stimulation currents can be delivered to the electrode device 1 and output via the electrode arrangement arranged on the electrode end 11 to stimulate the spinal cord R in the region of the spinal column W.

As can be seen from the sectional view of FIG. 2, in the embodiment shown the electrode device 1 is implanted with the electrode end 11 in the epidural space E in the region of the spinal column W of the patient P in such a way that the electrode end 11 is located in the region of the spinal cord R and can thus introduce stimulation currents in a directed manner into the spinal cord R in order to effect nerve stimulation in the region of the spinal cord R.

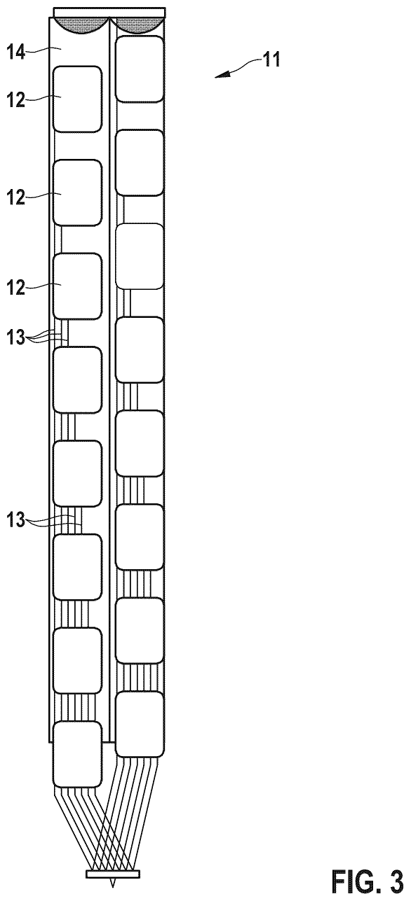

While the lead body 10, for example, comprises a circular (isodiametric) cross-section, the electrode device 1 is flattened in the area of the electrode end 11 which, as can be seen in FIG. 3, carries a plurality of contact elements 12, which may be evenly or unevenly spaced on the electrode end 11 in such a way that stimulation energy can be fed in a directed manner, for example, into the spinal cord R of a patient P.

As further illustrated in FIG. 3, each contact element 12 is connected to a supply line 13, wherein each contact element 12, for example, is connected to the stimulation device 2 via an associated, individual supply line 13 and thus may be supplied with stimulation energy via the stimulation device 2 to emit electrical signals. The supply lines 13 are jointly routed as a cable strand in the lead body 10 in an encapsulated manner to the stimulation device 2.

The contact elements 12 are arranged on a carrier 14 and are exposed with a surface facing outwards and can therefore come into contact with surrounding tissue when the electrode device 1 is implanted in a patient.

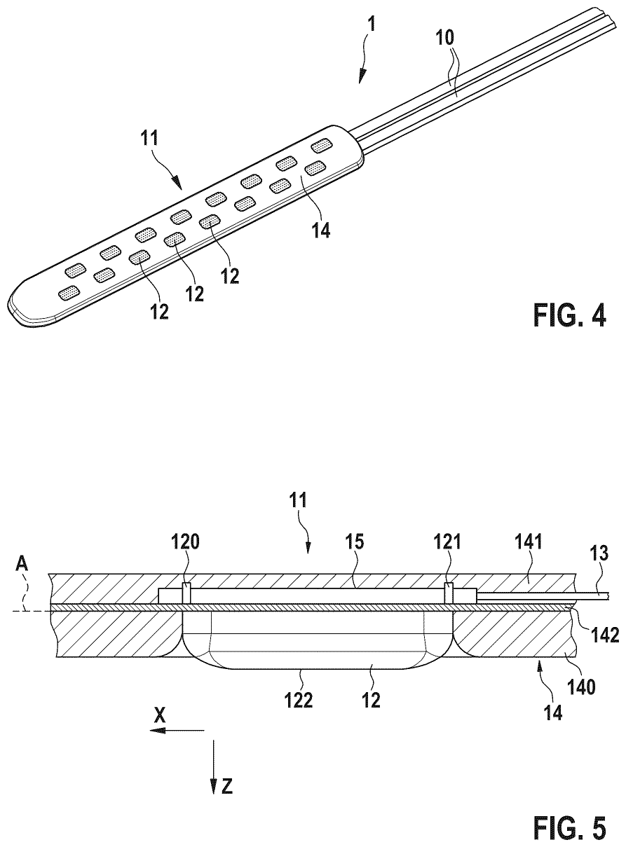

As visible from the embodiment of FIG. 4, multiple leads 10 may be connected to the electrode end 11, each lead 10 guiding electrical supply lines 13 for a portion of contact elements 12 arranged on the carrier element 14 of the electrode end 11.

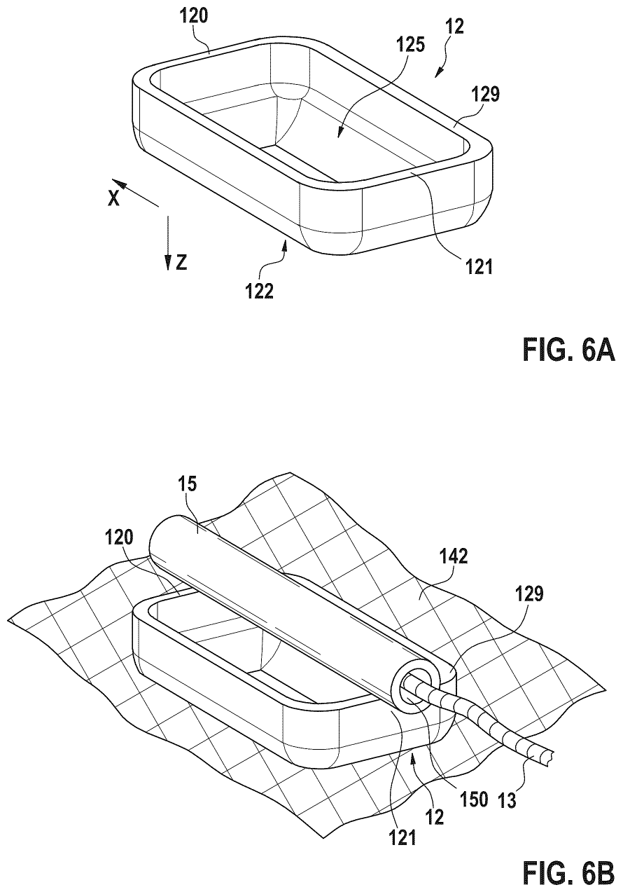

Referring now to FIG. 5, each contact element 12 is arranged and embedded in electrically insulating material 140, 141 of the carrier element 14, such that the contact element 12 at least partially is enclosed by the electrically insulating material 140, 141 of the carrier element 14. A contact portion 122 of the contact element 12 herein faces towards the outside and is exposed such that the contact portion 12 may come into contact with surrounding tissue in an implanted state of the electrode device 1.

As schematically shown in FIG. 5 and as it shall be further explained below, in one embodiment a reinforcement layer 142, for example, in the shape of a woven fabric is embedded within the electrically insulating material 140, 141 of the carrier element 14. The reinforcement layer 142 serves to increase the mechanical durability and form stability of the carrier element 14 and extends along a plane A within the flatly extending carrier element 14.

As also shall be further explained below, in one embodiment an electrical supply line 13 is connected to the contact element 12 by means of a connection element 15 which is arranged on and fastened to fastening sections 120, 121 integrally formed with the contact element 12. The arrangement of the connection element 15 on the contact element 12 herein is such that the reinforcement layer 142 and potentially in addition the electrically insulating material 140, 141 of the carrier element 14 extends through a space in between the contact portion 122 and the connection element 15 in a range in between the fastening sections 120, 121, such that the contact element 12 is mechanically held and locked to the carrier element 14 by engagement with the reinforcement layer 142.

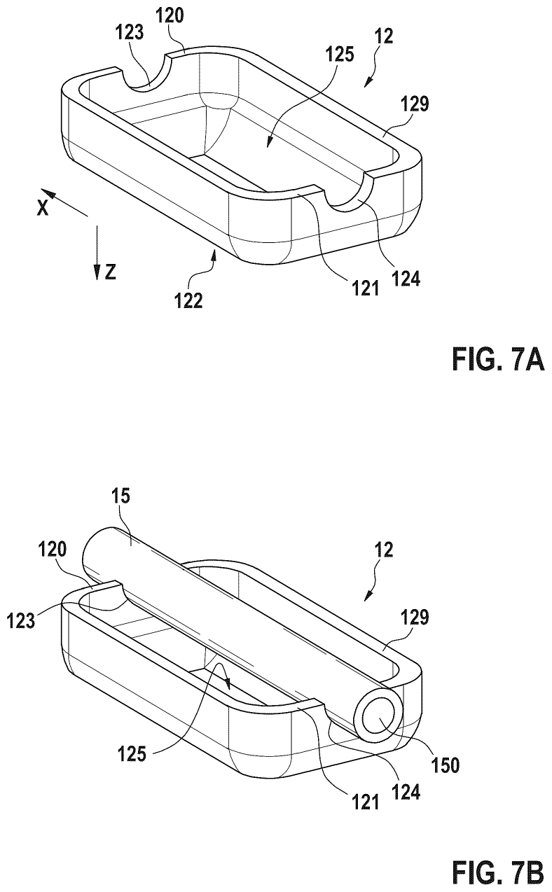

Referring now to FIGS. 6A, 6B, in one embodiment the contact element 12 is fabricated by a deep drawing process or by a machining process, the contact element 12 having the shape of a trough whose bottom forms the contact portion 122. The trough forms an inner space 125 and is bound by side walls of the contact element 12, an edge contour 129 circumferentially extending about the contact element 12 and facing in a direction opposite to the contact portion 122.

In the embodiment of FIGS. 6A, 6B, a connection element 15 in the shape of a sleeve, e.g., a crimp sleeve, is placed on the contact element 12 and fastened to the contact element 12, for example, by welding. The connection element 15 herein is placed on fastening sections 120, 121 formed by portions of the edge contour 129 on opposing side walls of the contact element 12, such that the connection element 15 extends across the contact element 12 in between the fastening sections 120, 121.

The connection element 15 forms an inner conduit 150 in which an associated electrical supply line 13 is received, the electrical supply line 13, for example, being connected to the connection element 15 by crimping or by welding.

As illustrated in FIG. 6B, the reinforcement layer 142 extends through the inner space 125 and hence through an opening formed in between the fastening sections 120, 121 and in between the connection element 15 and the contact portion 122 at the bottom of the trough-shaped contact element 12. The connection element 15 hence is placed on one side of the reinforcement layer 142, and the contact portion 122 on another, opposing side of the reinforcement layer 142. The reinforcement layer 142 hence engages with the contact element 12 such that the reinforcement layer 142 improves the mechanical stability and locational fixation of the contact element 12 with the connection element 15 arranged thereon on the carrier element 14.

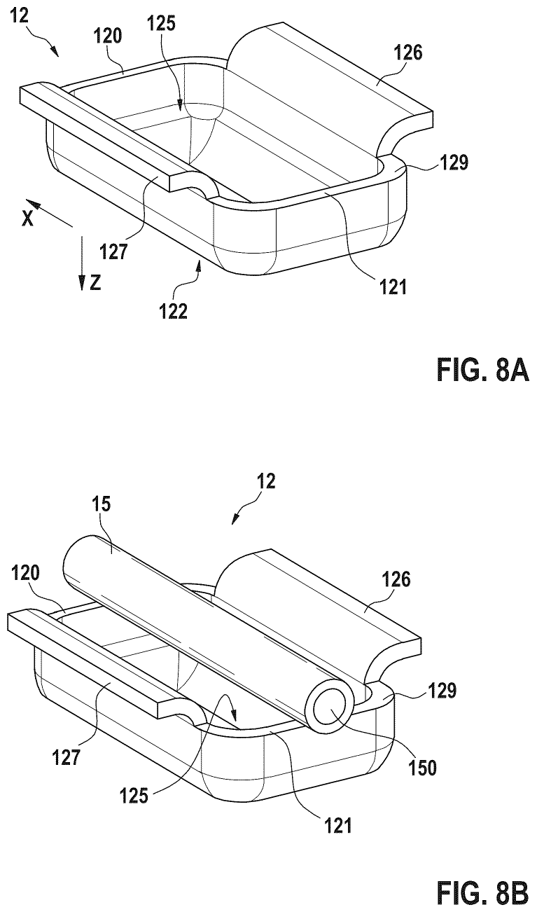

Referring now to FIGS. 7A, 7B, in one embodiment fastening openings 123, 124 are formed on the fastening sections 120, 121. The fastening openings 123, 124 serve to receive the connection element 15 therein, the fastening openings 123, 124 being formed by semicircular recesses in the embodiment of FIGS. 7A, 7B.

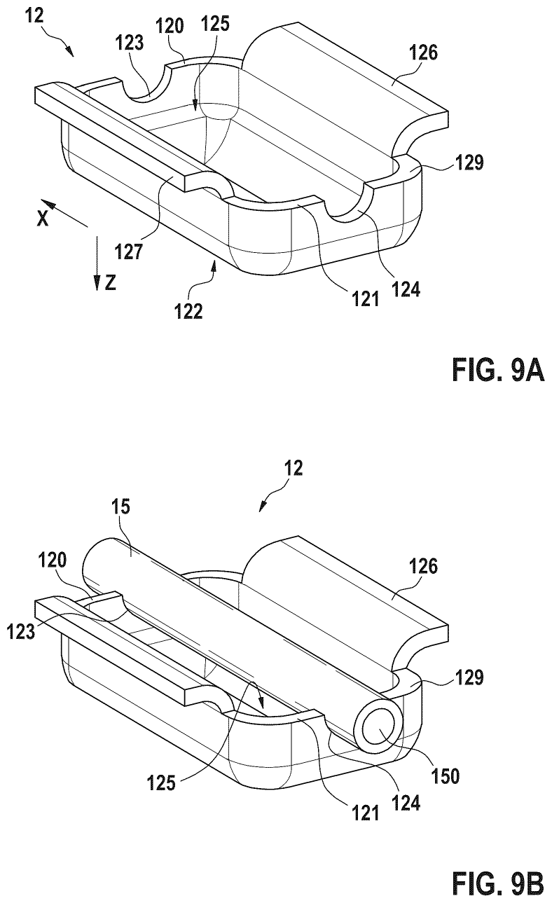

Referring now to FIGS. 8A, 8B, in another embodiment engagement elements 126, 127 are formed on the contact element 12, the engagement elements 126, 127 protruding from the edge contour 129 in a direction opposite the contact portion 122. The engagement elements 126, 127, in the shown embodiment, have the shape of wing elements extending outwards from the contact element 12 and serving to engage with the electrically insulating material 141 beyond the reinforcement layer 142 (see FIG. 5), in order to further improve the mechanical seating of the contact element 12 within the carrier element 14.

Other than that the contact element 12 of FIGS. 8A, 8B is similar to the contact element of FIGS. 6A, 6B, such that it also shall be referred to the description in relation to the embodiment of FIGS. 6A, 6B.

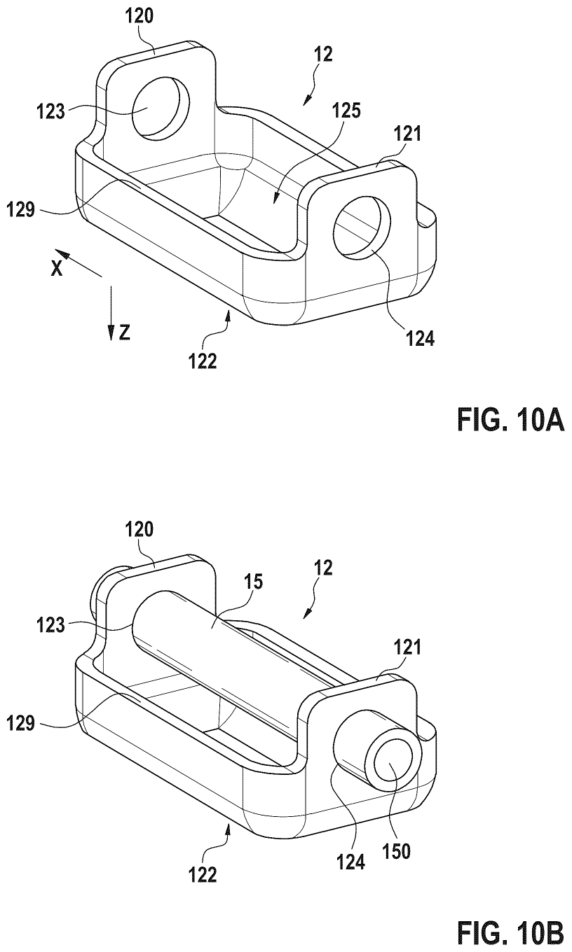

The embodiment of FIGS. 9A, 9B combines the embodiments of FIG. 7A, 7B and FIG. 8A, 8B. Namely, the contact element 12 in the embodiment of FIGS. 9A, 9B comprises engagement elements 126, 127, as present on the contact element 12 of FIGS. 8A, 8B. In addition, fastening openings 123, 124 in the shape of recesses are formed on the fastening sections 121, 122 for receiving the connection element 15.

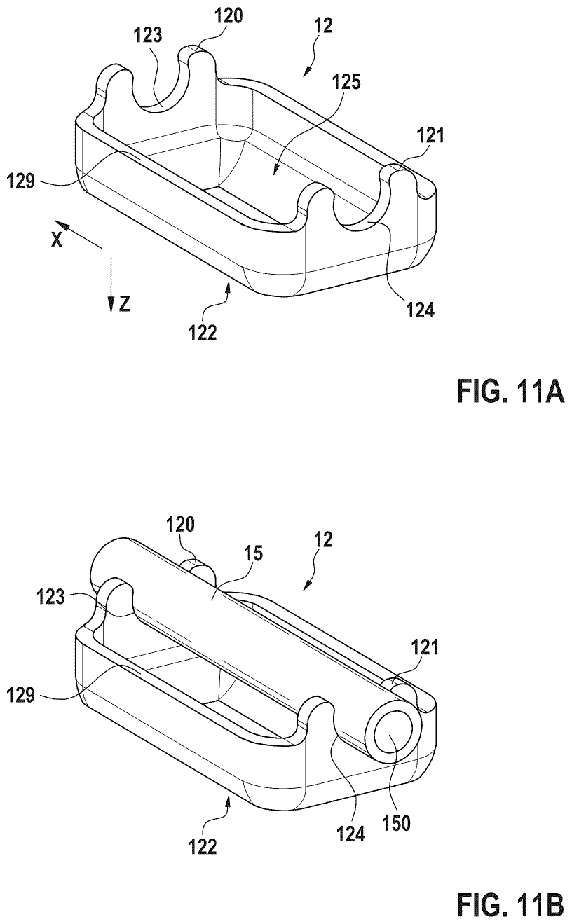

Referring now to FIGS. 10A, 10B, in one embodiment fastening openings 123, 124 with a circumferentially closed contour are formed in the fastening sections 120, 121, the fastening openings 123, 124 extending in a plane perpendicular to the length direction X and having a circular shape in that plane perpendicular to the length direction X. The connection element 15 extends through the fastening openings 123, 124 and across the contact element 12 and may, for example, be welded to the circumferential edge bounding the fastening openings 123, 124.

In the embodiment of FIGS. 10A, 10B, the fastening sections 120, 121 extend and protrude from (other portions of) the edge contour 129 of the contact element 12, the fastening sections 120, 121 being formed by tongues in which the fastening openings 123, 124 are formed.

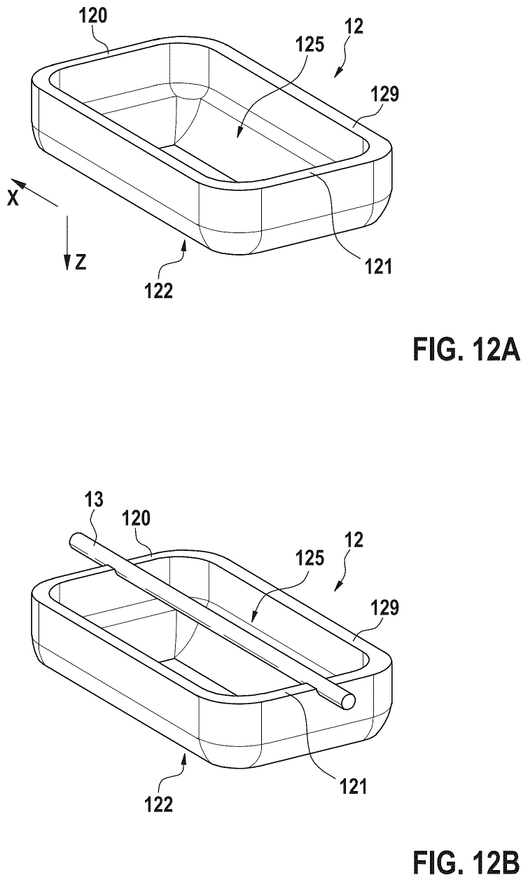

In yet another embodiment shown in FIGS. 11A, 11B, fastening openings 123, 124 are formed on the fastening sections 120, 121, the fastening openings 123, 124 being circumferentially opened, but extending about an angle larger than 180°, such that the connection element 15 may be clipped into the fastening openings 123, 124. Like in the embodiment of FIGS. 10A, 10B, the fastening sections 120, 121 protrude from the edge contour 129 along the height direction Z.

In the embodiments of FIGS. 6A, 6B to 11A, 11B a connection element 15 in the shape of a sleeve, for example a crimp sleeve, is used to establish a connection in between the electrical supply line 13 and the contact element 12. Hence, by means of the connection element 15 an indirect connection of the electrical supply line 13 to the fastening sections 120, 121 is established.

In other embodiments an electrical supply line 13 may be connected directly to the contact element 12.

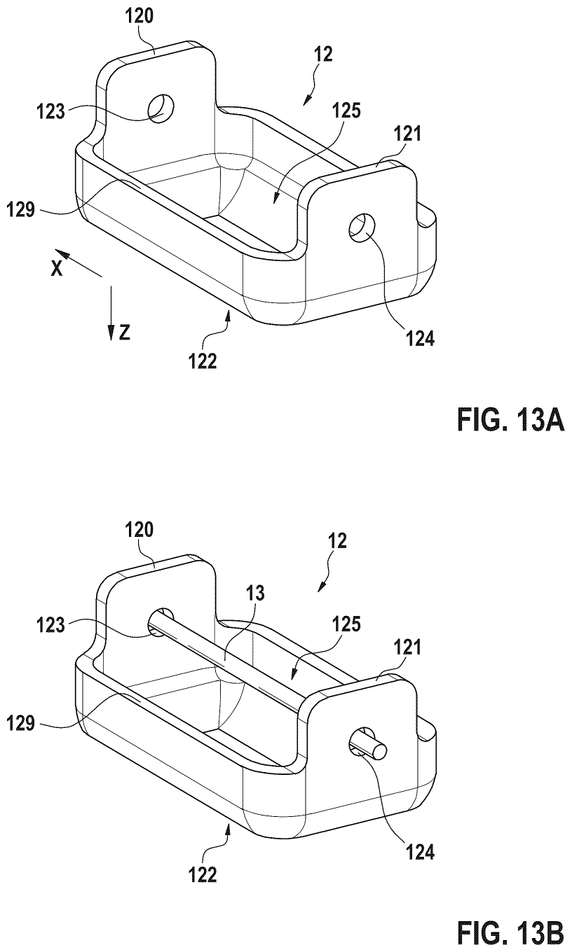

Referring now to FIGS. 12A, 12B, in one embodiment an electrical supply line 13 is directly placed on fastening sections 120, 121 formed by the edge contour 129, the contact element 12 having a similar shape as the contact element 12 according to the embodiment of FIGS. 6A, 6B. The electrical supply line 13 is connected to the fastening sections 120, 121, for example, by welding.

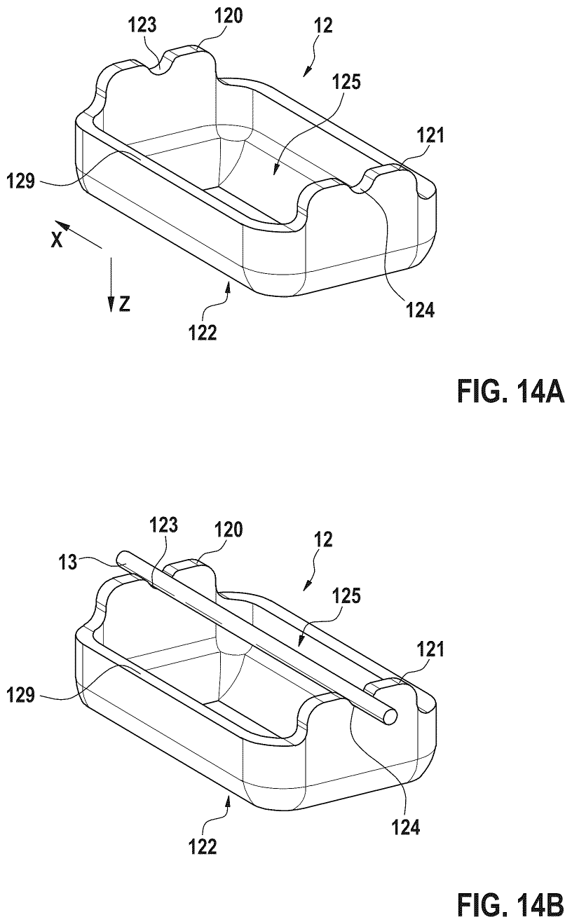

In another embodiment, the electrical supply line 13 may be received in and extend through fastening openings 123, 124 in fastening sections 120, 121 protruding from the edge contour 129 along the height direction Z, as this is shown in an embodiment in FIGS. 13A, 13B. Herein, in the embodiment of FIGS. 13A, 13B the fastening openings 123, 124 have a circumferentially closed, circular shape, similar to the embodiment of FIGS. 10A, 10B (the diameter of the fastening openings 123, 124 in the embodiment of FIGS. 13A, 13B however being adapted to the cross-sectional width of the electrical supply line 13).

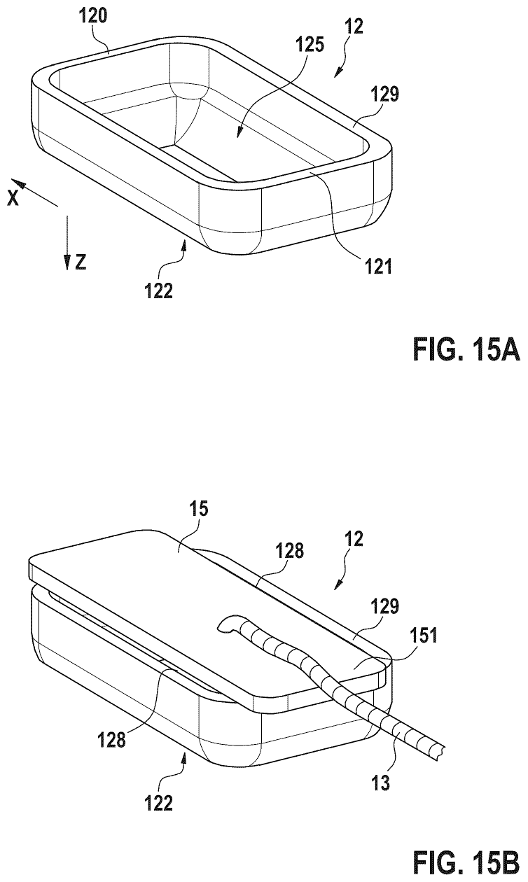

In yet another embodiment as shown in FIGS. 14A, 14B, the electrical supply line 13 is received in circumferentially opened fastening openings 123, 124 formed on fastening sections 120, 121 protruding from the edge contour 129 of the contact element 12.

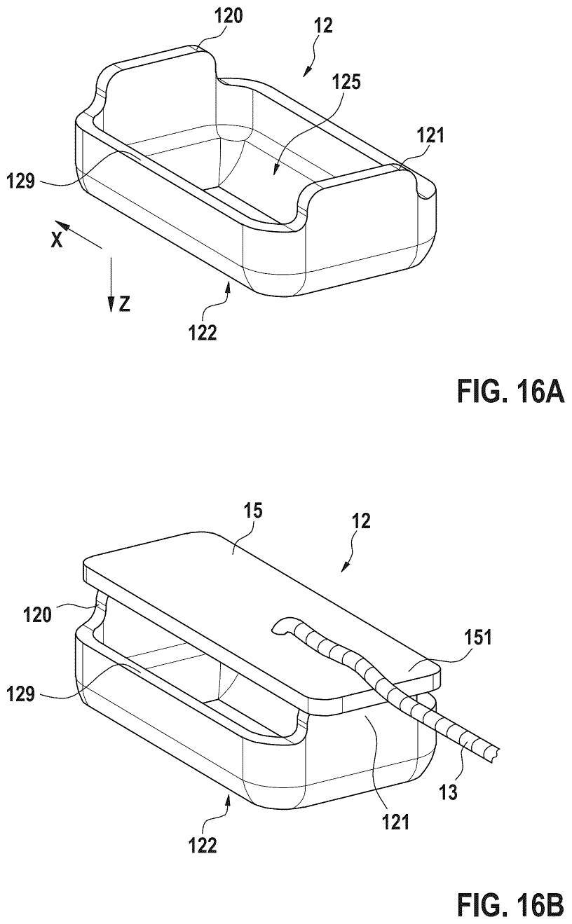

Referring now to FIGS. 15A, 15B, in another embodiment a connection element 15 having a plate body 151 is used to establish a connection between an electrical supply line 13 and the contact element 12. The contact element 12 herein has a shape similar to the contact element 12 of the embodiment of FIGS. 6A, 6B.

In the embodiment of FIGS. 15A, 15B, herein, the plate body 151 has a rectangular shape and flatly extends along a plane perpendicular to the height direction Z, the connection element 15 being fastened to fastening sections 120, 121 of the contact element 12 such that slit openings 128 are formed on both sides of the plate body 151 in between the plate body 151 and the associated outer side walls of the contact elements 12, as visible from FIG. 15B. Through these slit openings 128 a portion of the carrier element 14, for example insulating material 140, 141 and/or the reinforcement layer 142, may extend such that a portion of the carrier element 14 extends through the space 125 formed within the contact element 12 in order to improve the mechanical fixation of the contact element 12 on the carrier element 14.

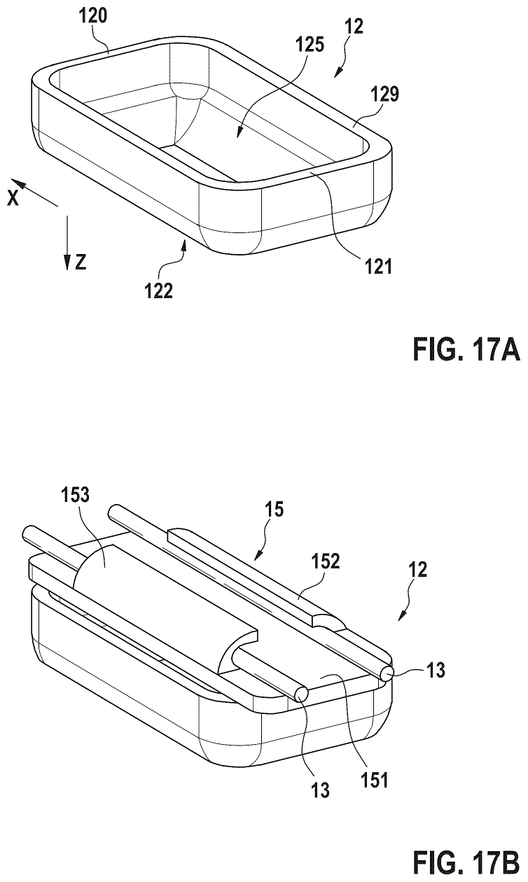

The connection element 15 may be, for example, welded to the fastening sections 120, 121. An electrical supply line 13 herein may be fastened to the connection element 15 on a side of the plate body 151 facing away from the contact element 12, for example by welding the electrical supply line 13 to the plate body 151.

Whereas in the embodiment of FIGS. 15A, 15B the fastening sections 120, 121 are formed by portions of the edge contour 129 and are arranged in the plane of the circumferential edge contour 129, in the embodiment of FIGS. 16A, 16B fastening sections 120, 121 protrude from other portions of the edge contour 129 in a direction opposite to the contact portion 122. Again, as visible from FIG. 16B, a connection element 15 having a plate body 151 is connected to the fastening sections 120, 121, the connection element 15 serving to establish a connection of an electrical supply line 13 to the contact element 12.

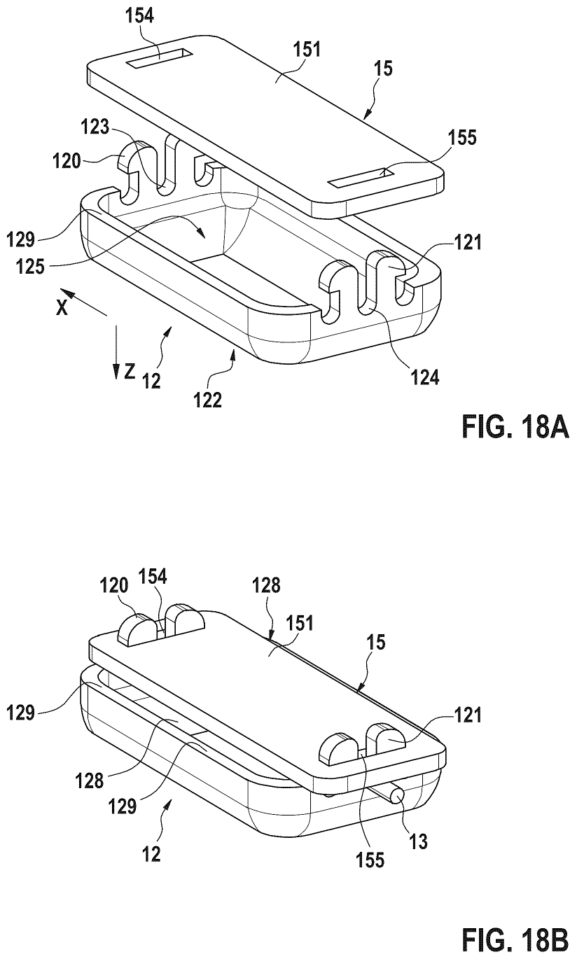

The embodiment of FIGS. 17A, 17B substantially matches the embodiment of FIGS. 15A, 15B, wherein receptacle elements 152, 153 are formed on the plate body 151 of the connection element 15 in order to receive electrical supply lines 13 therein. In the embodiment of FIGS. 17A, 17B, herein, two supply lines 13 are connected to the connection element 15 in order to establish an electrical connection to the contact element 12. In the embodiment of FIGS. 17A, 17B, also only one supply lines 13 can be connected to the connection element 15 in order to establish an electrical connection to the contact element 12. In this way one or another side of the connection element 15 can be chosen during assembly.

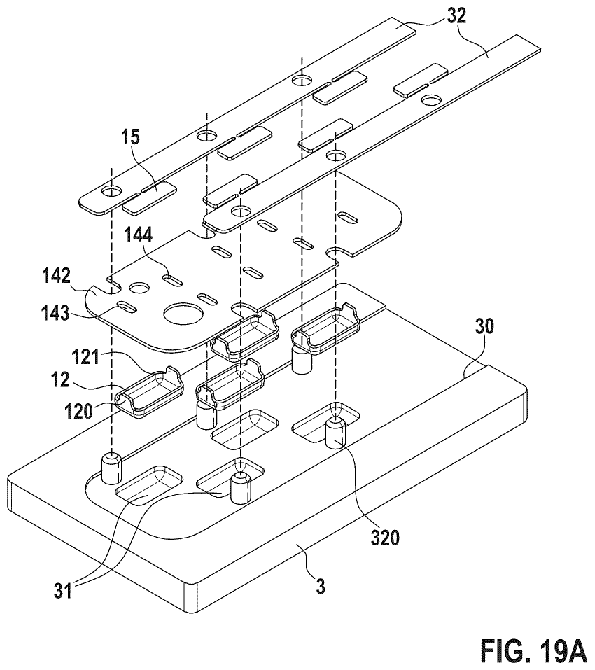

Referring now to FIGS. 18A, 18B, in one embodiment the fastening sections 120, 121 are configured to establish detent connections to a connection element 15 having a plate body 151, engagement openings 154, 155 being formed in the plate body 151 for engaging with the fastening sections 120, 121.

In a connected state, as shown in FIG. 18B, the fastening sections 120, 121 are in engagement with the engagement openings 154, 155, such that the connection element 15 is mechanically connected to the contact element 12.

In the embodiment of FIGS. 18A, 18B, fastening openings 123, 124 are formed on the fastening sections 120, 121, an electrical supply line 13 being received within the fastening openings 123, 124. The electrical supply line 13 is placed in the fastening openings 123, 124 such that the electrical supply line 13 extends along a side of the plate body 151 of the connection element 15 pointing towards the contact portion 122, the electrical supply line 13 hence being received in between the contact element 12 and the connection element 15.

The connection element 15 herein may, for example, establish a clamping connection of the electrical supply line 13 to the contact element 12, wherein in addition the electrical supply line 13 may, for example, be welded to the contact element 12 at the fastening sections 120, 121.

In all of the embodiments as described above a portion of the carrier element 14 extends through a space 125 formed in between the electrical supply line 13 (or a connection element 15) and the contact portion 122 at the bottom of the contact element 12 and in between the fastening sections 120, 121. The portion of the carrier element 14 herein may be formed by electrically insulating material 140, 141 filled into the space 125 during a molding process when fabricating the electrode device 1, and/or may be a reinforcement layer 142, for example, comprising a woven fabric embedded in the electrically insulating material of the carrier element 14. By having the portion of the carrier element 14 reach through the opening bound by the electrical supply line 13 (or the connection element 15), the contact portion 122 and the fastening sections 120, 121 at the side walls of the contact elements 12, the contact element 12 is locked to the carrier element 14 and hence is held on the carrier element 14 in a mechanically stable manner.

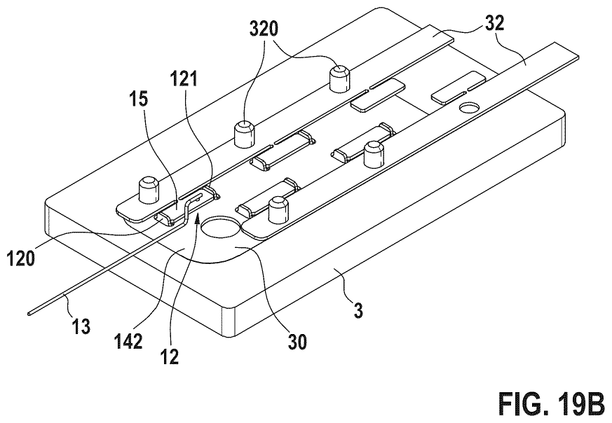

Referring now to FIGS. 19A, 19B, during the fabrication of the electrode device 1 the contact elements 12, the connection element 15 and the electrical supply lines 13 are assembled using a positioning tool 3 with a cavity 30. For the assembly, contact elements 12 are arranged in receptacles 31 within the cavity 30. A reinforcement layer 142 may be placed on the contact elements 12 such that, e.g., protruding fastening sections 120, 121 of the contact elements 12 reach through openings 143, 144 formed within the reinforcement layer 142. Connection elements 15 are then placed on the fastening sections 120, 121 at a side of the reinforcement layer 142 facing away from the contact elements 12, such that the reinforcement layer 142 is received in between the contact elements 12 on the one side and the connection element 15 on the other side of the reinforcement layer 142. To improve the handling of the connection elements 15, the connection elements 15 are initially connected to a connection elements carrier 32 to simplify the simultaneous positioning of the connection elements 15. The positioning of the connection elements 15 is supported by positioning studs 320.

The connection elements 15 each may, for example, be fixed to the fastening sections 120, 121 of an associated contact element 12 by welding. In addition, an electrical supply line 13 may be connected to each connection element 15, e.g., by welding, as this is shown in FIG. 19B. After the assembly process, the connection elements 15 can be separated from the connection elements carrier 32, for example, using a Laser.

The electrical supply line 13 may, for example, be formed from a DFT wire (Mp35N/Ag), having a diameter, for example, in between 0.1 mm and 0.3 mm, for example 0.14 mm. In addition, the electrical supply line 13 may, for example, be covered with ETFE as an insulation cover.

The carrier element 14 may be molded by a molding process surrounding the pre assembled inner parts of the electrode end 11, comprising the contact elements 12, the connection elements 15, the electrical supply lines 13 and the reinforcement layer 142.

The electrically insulating material 140, 141 of the carrier element 14 may, for example, be a polyurethane or a silicone material or a combination of both materials as well as an other thermoplastic material.

The carrier element 14 may, for example, have an overall thickness not larger than 3 mm, preferably not larger than 2 mm.

When using a connection element 15 in the shape of a crimp sleeve, the connection element 15 may be formed, e.g., from a Pt/Ir material or from MP35 and may form a conduit having a diameter adapted to receive the electrical supply line 13 therein, for example in a range between 0.11 mm to 0.35 mm, for example in a range between 0.15 mm to 0.19 mm.

The contact portion 122 may have an area, for example, of 2 mm×3.5 mm, which is exposed towards the outside for establishing a contact to surrounding tissue. The contact element 12 may, for example, be made from a biocompatible material like gold, platinum, MP35N or a Pt/Ir material.

The idea of the present invention is not limited to the embodiments described above, but may be implemented in an entirely different fashion.

In particular when using a connection element to connect the electrical supply line to the associated contact element, tolerances can be adjusted and the electrical supply line can be variably connected to the contact element as desired.

When using a connection element in the shape of a crimp sleeve it may not be required to remove an insulating cover of the electrical supply line, a reliable electric connection being established by establishing a crimping connection in between the electrical supply line and the crimp sleeve.

A process for manufacturing an electrode device of the kind described herein may be easily automatable.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable electrode device
10 Lead body
11 Electrode end
12 Contact element
120, 121 Connection section
122 Contact portion
123, 124 Fastening opening
125 Inner space
126, 127 Engagement element
128 Slit openings
129 Edge contour
13 Supply line
14 Carrier element
140, 141 Insulating material
142 Portion (reinforcement layer)
143, 144 Openings
15 Connection element
150 Conduit (lumen)
151 Plate body
152, 153 Receptacle element
154, 155 Engagement opening
2 Stimulation device
20 Connector block
3 Positioning tool
Cavity
31 Receptacle
32 Connection elements carrier
320 Positioning studs
A Plane of extension
E Epidural space
P Patient
R Spinal cord
W Spinal column
X Length direction
Z Height direction

1. A medical electrode device for implantation into a patient, comprising:
a carrier element (14) being formed from an electrically insulating material,
at least one contact element arranged on the carrier element and comprising a contact portion for coupling to tissue in an implanted state of the medical electrode device, and
at least one electrical supply line connected to the at least one contact element,
wherein the at least one contact element comprises a first fastening section and a second fastening section, the first fastening section and the second fastening section being arranged at a distance with respect to each other along a length direction and the at least one electrical supply line being connected to the at least one contact element at the first fastening section and the second fastening section such that the at least one electrical supply line is arranged at a distance with respect to the contact portion along a height direction oriented perpendicularly to the length direction,
wherein a portion of the carrier element extends through a space in between the contact portion and the at least one electrical supply line.

2. The medical electrode device according to claim 1, wherein the contact portion faces outside in a first direction along the height direction, wherein the at least one contact element comprises an edge contour facing in a second direction opposite the first direction, the first fastening section and the second fastening section being formed on the edge contour.

3. The medical electrode device according to claim 2, wherein at least one of the first fastening section and the second fastening section protrudes from the edge contour along the second direction.

4. The medical electrode device according to claim 2, wherein the at least one contact element comprises at least one engagement element protruding from the edge contour and engaging with the electrically insulating material of the carrier element.

5. The medical electrode device according to claim 4, wherein the first fastening section and the second fastening section are formed on a first portion and a second portion of the edge contour arranged at a distance with respect to each other along the length direction, wherein the at least one engagement element is arranged on a third portion of the edge contour extending in between the first portion and the second portion along the length direction.

6. The medical electrode device according to claim 1, wherein at least one of the first fastening section and the second fastening section comprises a fastening opening in which the at least one electrical supply line is arranged.

7. The medical electrode device according to claim 6, wherein the fastening opening is circumferentially closed or is circumferentially opened.

8. The medical electrode device according to claim 1, wherein a connection element configured to connect the at least one electrical supply line to the first fastening section and the second fastening section, the connection element being fastened on the first fastening section and the second fastening section.

9. The medical electrode device according to claim 8, wherein the connection element comprises a conduit in which the at least one electrical supply line is received.

10. The medical electrode device according to claim 8, wherein the connection element comprises a plate body configured to fasten the at least one electrical supply line.

11. The medical electrode device according to claim 10, wherein the at least one electrical supply line is arranged on a side of the plate body facing away from the contact portion.

12. The medical electrode device according to claim 10, wherein the at least one electrical supply line is arranged on a side of the plate body facing towards the contact portion.

13. The medical electrode device according to claim 1, wherein said portion of the carrier element is a reinforcement layer embedded in the electrically insulating material of the carrier element.

14. The medical electrode device according to claim 13, wherein said reinforcement layer comprises a woven fabric.

15. A method for fabricating a medical electrode device for implantation into a patient, the method comprising:
forming a carrier element from an electrically insulating material such that at least one contact element is at least partially embedded in the electrically insulating material of the carrier element, the at least one contact element comprising a contact portion for coupling to tissue in an implanted state of the medical electrode device, and
connecting at least one electrical supply line to the at least one contact element, wherein the at least one contact element comprises a first fastening section and a second fastening section, the first fastening section and the second fastening section being arranged at a distance with respect to each other along a length direction and the at least one electrical supply line being connected to the at least one contact element at the first fastening section and the second fastening section such that the at least one electrical supply line is arranged at a distance with respect to the contact portion along a height direction oriented perpendicularly to the length direction,
wherein said forming the carrier element includes: forming the carrier element such that a portion of the carrier element extends through a space in between the contact portion and the at least one electrical supply line.
